(19) United States
(12) Patent Application Publication
(10) Pub. No.: US 2020/0061315 A1
(43) Pub. Date: Feb. 27, 2020

(54) VENTILATING APPLIANCE

(71) Applicant: R. Cegla GmbH & Co. KG, Montabaur (DE)

(72) Inventors: Andrea Ebinger, Montabaur (DE); Ulrich Cegla, Montabaur (DE)

(21) Appl. No.: 16/545,694

(22) Filed: Aug. 20, 2019

(30) Foreign Application Priority Data
Aug. 23, 2018 (DE) 10 2018 120 533.3

(51) Int. Cl. A61M 16/00; A61M 16/06; A61M 16/20

(52) U.S. Cl. CPC A61M 16/0012; A61M 16/06; A61M 16/208; A61M 2016/0036; A61M 16/0488; A61M 2202/0208; A61M 2230/432; A61M 2016/0027; A61M 2210/0618

(57) ABSTRACT

For a ventilating appliance (1) consisting of a ventilating device (2) having an air outlet channel (3) and an air inlet channel (4), two hoses (5, 6) which are connected to one of the two channels (3, 4) of the ventilating device (2), an air distributor (7), on which three openings (8, 9, 10) are provided, which are coupled to one of the two hoses (5, 6) of the ventilating device (2) and to a tube (11) which can be inserted into the mouth or nose space (22) of a living being (21), shall the ventilating appliance (1) be improved that a patient to be ventilated undergoes a release of mucus and its removed from the lung area with cost-effective measures, without the need for foreign objects or switching off the ventilating appliance (1). This is solved in that a respiratory tract therapy device (31) is inserted in the inhalation hose (5), in that an air flow (12) is generated by the ventilating device (2), through which an intermittent air pressure fluctuation arises with the respiratory tract therapy device (31), and in that the air pressure fluctuations generated in this way are passed on or transmitted in the lung region (23) of the living being (21).

### REFERENCE TO PENDING PRIOR PATENT APPLICATION

This patent application claims benefit of German Patent Application No. 10 2018 120 533.3, filed Aug. 23, 2018, which patent application is hereby incorporated herein by reference.

### FIELD OF THE INVENTION

The invention refers to ventilating appliances in general, and more particularly to a ventilating appliance comprising a ventilating device having an air outlet channel and an air inlet channel, two hoses which are connected to one of the two channels of the ventilating device, and an air distributor on which three openings are provided, which are coupled to one of the two hoses of the ventilating device and to a tube which can be inserted into the mouth or nose space of a living being.

### BACKGROUND OF THE INVENTION

Such ventilating appliances have been known for many years and are usually used for living beings, especially people, who can no longer breathe independently. The ventilating device assigned to the ventilating appliance generates an intermittent air flow, which can also be enriched with oxygen and other gases, for example. This air flow is passed through a ventilating tube and an air distributor to a tube connected to it, which is inserted into the lung area of the patient. The breathing air generated by the ventilating device thus reaches the patient's lungs. When the patient exhales, the consumed breathing air is sucked through the tube into the air distributor and from there back into the ventilating device.

In such treatment situations, it has been found that the patient's lung area becomes mucous, especially if the patient has to be ventilated for a relatively long period of time. However, occluding the lungs can cause infections and breathing difficulties that can be life-threatening.

Although EP 2 489 413 B1 discloses a therapy device for the treatment of respiratory diseases in patients, the respiratory tract therapy devices of prior art can only be used for patients who can breathe independently. Such therapy devices cannot be used for patients who can no longer breathe independently and must therefore be connected to a ventilating device of prior art. The air resistances generated within the therapy device during inhalation and/or exhalation cause oscillating air pressure fluctuations, which are transmitted to the bronchial and lung areas of the patient and have an expectorant effect there. As a result, the mucus released from the lungs can be degenerated or removed independently. The so-called PEP or OPeP therapy devices, by means of which positive expiratory pressure (PEP) or oscillating positive expiratory pressure (OPEP) ventilating treatments can be carried out, generate a positive and negative ventilating pressure, through which the mucus deposited in the lungs can be released and transported out of them.

The combination of the known respiratory tract therapy devices, which work according to the treatment principle described above, and the known ventilating appliances have not been disclosed. Rather, the mucus in the lungs of the patient who is not breathing independently is loosened surgically or by other medical measures and transported away, as a result of which, however, a high risk of infection or respiratory arrest exists, because the patient is uncoupled from the ventilating device—at least for a short time —and foreign objects must be used for releasing the mucus and removing it.

### SUMMARY OF THE INVENTION

It is therefore the task of the present invention to develop the aforementioned ventilating appliances in such a way that a patient to be ventilated undergoes a release of mucus and its removal from the lung area with cost-effective measures, without the need for foreign objects or switching off the ventilating appliance.

This task is accomplished according to the invention by the features of the characterizing part of patent claim 1.

Further advantageous embodiments of the invention are described in the subordinate claims.

The fact that a respiratory tract therapy device is used in the inhalation tube, that an air flow is generated by the end in the respiratory tract therapy device, through which an intermittent air pressure fluctuation arises in the respiratory tract therapy device, and that the air pressure fluctuations generated in this way are passed on or transmitted in the bronchial and lung region of the living beings, means that both positive and negative air pressures act on the pulmonary alveoli, as a result of which mucus deposits are released and removed. Consequently, the ventilating appliance according to the present invention can also be used for living beings which cannot breathe independently but require medical assistance in the form of artificial or mechanical ventilation. Such a medical treatment measure advantageously leads to the fact that the patients connected to the ventilating appliance have no or only very little mucus deposition in their bronchial and lung areas, since the permanent air pressure fluctuations caused by the air flow generated by the ventilating device and the design of the respiratory tract therapy device cause the mucus to be released and at the same time these mucus particles can be breathed out or exhaled by the patient.

The respiratory tract therapy device can consist of a curved tube in which a freely movable hose is fixed, or it can be designed as a valve in the form of a throttle valve or a ball. A decisive factor for the generation of positive and negative air pressure fluctuations within the respiratory tract therapy device is that the respiratory air uniformly generated by the ventilating device and pressed into the tube of the respiratory tract therapy device has an oscillating pressure level, resulting in corresponding air pressure fluctuations within the patient's lungs or bronchi. The oscillating air bridge generated in this way causes foreign particles, in particular mucus, to separate from the alveoli and be transported outwards with the extracted breathing air.

Since a patient often does not permanently need the oscillating air pressure fluctuations generated by the respiratory tract therapy device, it is advantageous if the respiratory tract therapy device can be removed or installed from the ventilating hose of the ventilating appliance without loss of time and without complications. For this purpose, the two ends of the tube piece are equipped with corresponding fastening adapters which can be plugged together into corresponding fastening adapters associated with or attached to the ventilating hose so that the tube piece can be clipped or threaded together with the free ends of the ventilating hose.

In an advantageous embodiment, the fastening adapters on the ventilating hose are configured in such a way that they can be assembled with an airtight seal when the respiratory tract therapy device is removed from the ventilating hose.

A particularly advantageous further embodiment of the present invention is to provide a bypass line parallel to the respiratory tract therapy device, which can be controlled via a valve. When the valve is set to an open position, the air generated by the ventilating device flows directly through the bypass line to the patient. When the valve is closed, the air generated by the ventilating device flows through the respiratory tract therapy device, creating the desired intermittent variations in air pressure that enter the bronchi or lungs of the living being and cause the mucus to be released.

### BRIEF DESCRIPTION OF THE DRAWINGS

The drawings show two examples of a ventilating appliance according to the present invention, which are explained in more detail below. In the drawings,

FIG. 1 shows a first configuration variant of a ventilating appliance consisting of a ventilating device and two ventilating hoses attached thereto in which a respiratory tract therapy device is inserted, of an air distributor to which the ventilating hoses are connected and a tube associated with the air distributor, which tube is inserted into the lung region of a living being, in side view,

FIG. 2 shows an enlarged section of the ventilating appliance according to FIG. 1 with a respiratory tract therapy device inserted into the ventilating hose, in a sectional view,

FIG. 3a shows the respiratory tract therapy device attached to the ventilating hose according to FIG. 2,

FIG. 3b shows the ventilating hose disconnected from the respiratory tract therapy device according to FIG. 3a,

FIG. 4a shows a second embodiment variant of a ventilating appliance consisting of a ventilating device and a ventilating and exhalation hose which is looped around the exhalation hose, and of a respiratory tract therapy device inserted into the ventilating hose, in a sectional view,

FIG. 4b shows the connection between the ventilating hose and the respiratory tract therapy device according to FIG. 4 along the intersection IVb-IVb, and

FIG. 5 shows the appliance according to FIG. 1 with a bypass line and a valve by means of which the respiratory tract therapy device can be connected to or disconnected from the ventilating device.

### DETAILED DESCRIPTION OF THE PREFERRED EMBODIMENTS

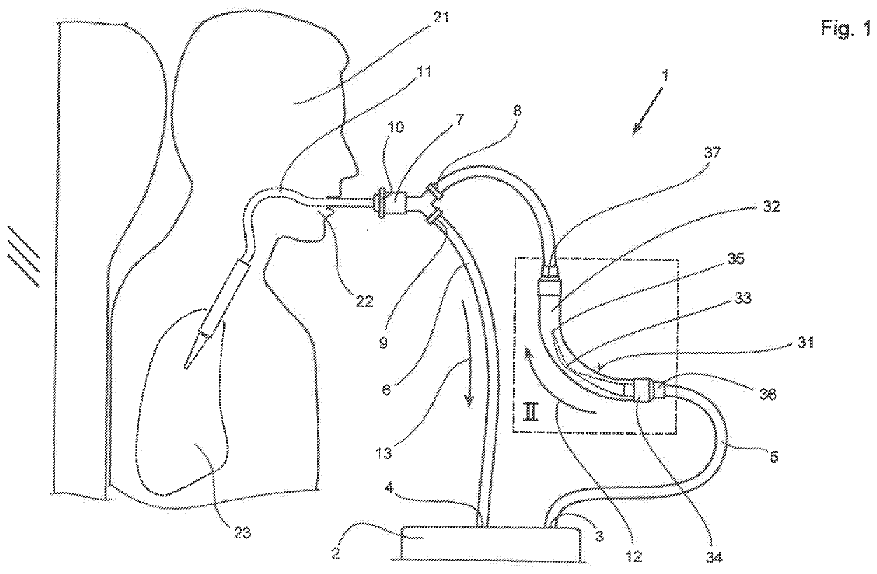

FIG. 1 shows a ventilating appliance 1 through which a living being 21, for example a human being or an animal, is to be ventilated, since the living being 21 can no longer breathe on its own.

The ventilating appliance 1 consists of a ventilating device 2, through which breathing air is generated and directed towards an air outlet channel 3. In addition, an air inlet channel 4 is provided on the ventilating device 2. A ventilating hose 5 is attached to the air outlet channel 3 and an exhalation hose 6 is attached to the air inlet channel 4. The ventilating hose 5 can also be referred to as the inhalation leg or coaxial hose.

In addition, an air distributor 7 with three openings 8, 9 and 10 is assigned to the ventilating appliance 1. The ventilating hose 5 is connected to the opening 8, the exhalation hose 6 to the opening 9 and a tube 11 to the opening 10. The tube 11 is introduced into the lung area 23 through the mouth cavity 22 of the living being 21, so that the breathing air 18 produced by the ventilating device 2 is pressed into the lung 23 through the ventilating hose 5, the air distributor 7 and the tube 11, in which case the patient 21 is artificially ventilated. The consumed breathing air 13 is sucked out of the lung 23 within a given cycle by the ventilating device 2 and the air passes via the tube 11 and the air distributor 7 into the exhalation hose 6.

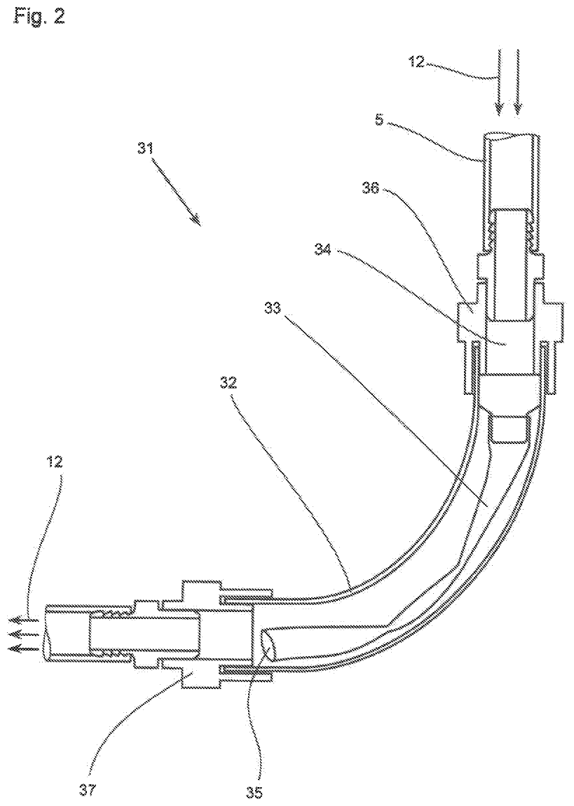

FIG. 2 shows a respiratory tract therapy device 31 installed in the ventilating hose 5. The respiratory tract therapy device 31 consists of a curved tube 32 in which a hose 33 is provided. The hose 33 is held with one end at the breathing air inlet side 34 of the respiratory tract therapy device in a position-oriented manner on the tube 22 in such a way that the breathing air 12 pressed into the tube 32 reaches the inside of the hose 33. The opposite free end of the hose 33 can therefore oscillate freely inside the tube 32. Due to the curvature of the tube 32 and the movement possibilities of the hose 33, an oscillating air pressure fluctuation occurs which passes from the tube 32 into the continuation of the ventilating hose 5 and thus reaches the lung 23 of the patient 21 from the air distributor 7 and the tube 11.

These air pressure fluctuations lead to the release of the mucus in the lung 23 of the patient 21, because the positive and negative air pressure releases the possibly existing mucus in the lung 23, so that freely movable mucus particles develop. The negative pressure generated by the ventilating device is used to suck out the consumed breathing air 13 and causes these mucus particles to be carried out of the lung 23 and transported away with its help.

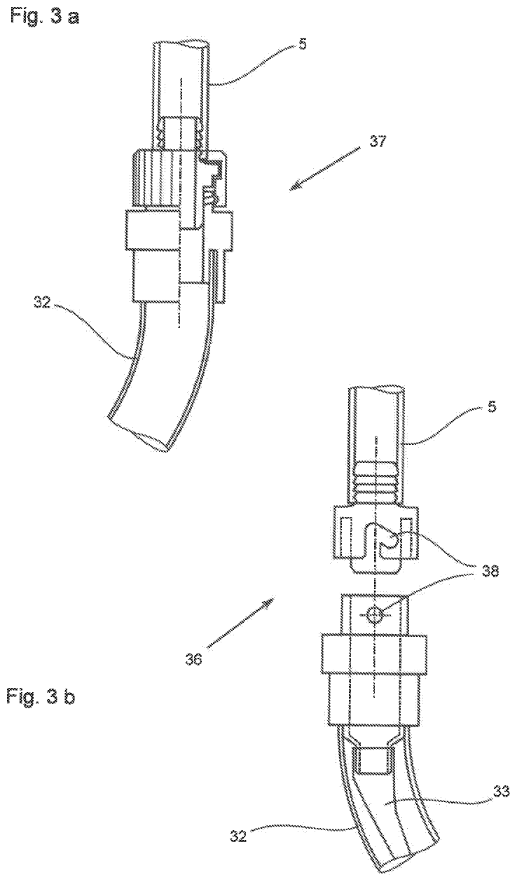

FIGS. 3a and 3b show how the tube 32 can be attached to the ventilating hose 5 of the ventilating appliance 1. For this purpose, fastening adapters 36 and 37 are provided at each free end of the tube 32, which are firmly connected to the tube 32 creating an airtight seal. Both fastening adapters 36 and 37 are provided with a push-button or a pin which can be inserted into an essentially L-shaped groove and into which they engage or are clipped in according to the method of a bayonet closure 38 or an undercut.

The tube 32 can also be attached to the ventilating hose 5 by means of an external and internal thread. The only factors which are decisive for the function are that the respiratory tract therapy device 31 can be connected to the ventilating hose 5 with an airtight seal and that the respiratory tract therapy device 31 can be removed from or connected to the ventilating hose 5 without delay and without complications.

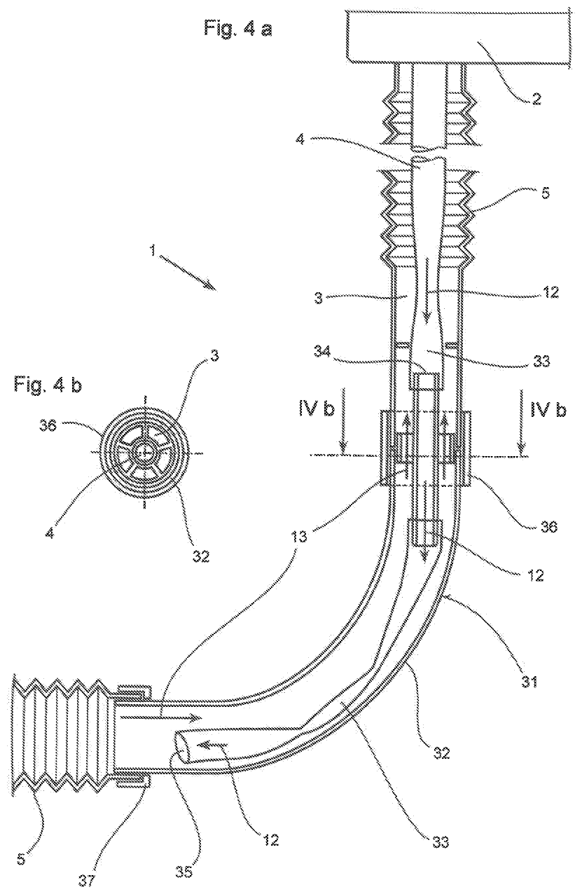

According to FIGS. 4a and 4b, the ventilating hose 5 is located inside the exhalation hose 6 in such a way that a space is created between them through which the consumed breathing air 13 is sucked by the ventilating device 2.

The respiratory tract therapy device 31 inserted into the ventilating hose 5 is thus subjected to the air flow forced out by the ventilating device 2 and the hose 33 vibrates inside the tube 32. When the consumed breathing air 13 is sucked out, the hose 33 closes completely, so that this breathing air 13 flows along the side of the hose 33.

The valve 19 channels the oxygen supply from ventilating device 2 directly to the patient via the bypass line 18 or draws the oxygen flow through the respiratory tract therapy device 31. The valve 19 can be moved manually from the outside to these two positions and thus forms a kind of branch for the oxygen flow.

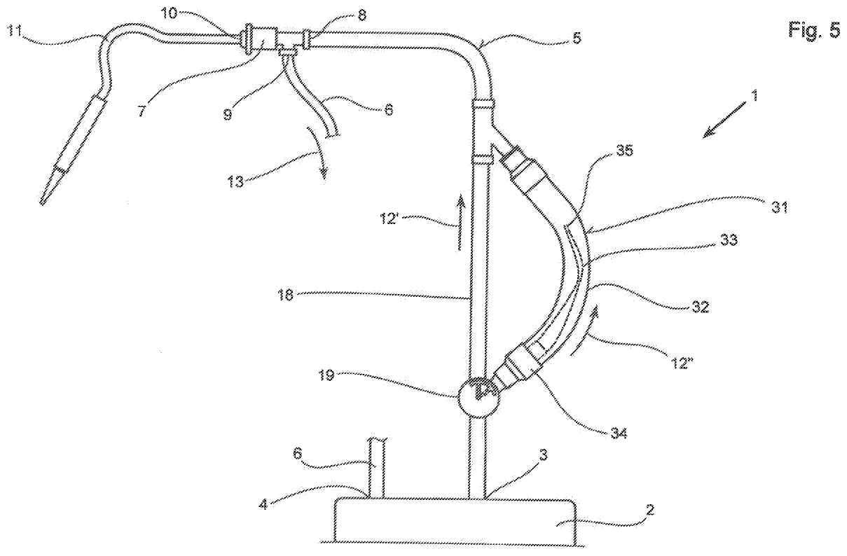

FIG. 5 shows how the respiratory tract therapy device 31 is to be decoupled from the ventilating circuit via a bypass line 18 when a valve 19 inserted into it is closed. As soon as the valve 19 closes the air access into respiratory tract therapy device 31, oxygen from the ventilating device 2 flows directly through a bypass line 18 and the respiratory tract therapy device 31 does not function. If the patient should also be provided with the effect of the respiratory tract therapy device 31, the valve 19 must be set so that the oxygen flows from the ventilating device 2 into the tube 32 of the respiratory tract therapy device 31 and causes the hose 33 to oscillate.

The appliance 1 according to the present invention generates an intermittent air pressure fluctuation in all the embodiment variants described, which are transmitted to the bronchi or lung areas of the living being 21 and cause the mucus there to be released.

What is claimed is:

1. A ventilating appliance (1) consisting of:
a ventilating device (2) having an air outlet channel (3) and an air inlet channel (4),
two hoses (5, 6) which are connected to one of the two channels (3, 4) of the ventilating device (2), and
an air distributor (7), on which three openings (8, 9, 10) are provided, which are coupled to one of the two hoses (5, 6) of the ventilating device (2) and to a tube (11) which can be inserted into the mouth or nose space (22) of a living being (21),
characterised in that,
a respiratory tract therapy device (31) is inserted in the inhalation hose (5), in that an air flow (12) is generated by the ventilating device (2), through which an intermittent air pressure fluctuation arises with the respiratory tract therapy device (31), and in that the air pressure fluctuations generated in this way are passed on or transmitted in the lung region (23) of the living being (21).

2. The device according to claim 1,
characterised in that,
the respiratory tract therapy device (31) consists of a curved tube (32) which is integrated in an airtight manner in the ventilating hose (5) or is connected to the latter, in that a flexible hose (33) is fastened to the respiratory air inlet side (34) of the respiratory tract therapy device (31), and in that the opposite end (35) of the hose (33) oscillates in the tube (32) and through the pressed-in air flow (12).

3. The device according to claim 1,
characterised in that,
the respiratory tract therapy device (31) consists of a curved tube (32) which is integrated in an airtight manner in the ventilating hose (3) or is connected to the latter, in that a valve in the form of a throttle valve or a ball is inserted inside the tube (32), which valve is set into oscillating vibration by the pressed-in air flow (12).

4. The device according to claim 1,
characterised in that,
two fastening adapters (36, 37) are provided on the ventilating hose (5), on which the respiratory tract therapy device (31) is locked in the manner of a bayonet closure (38), a thread or by means of push buttons.

5. The device according to claim 4,
characterised in that,
the two fastening adapters (36, 37) can be locked or fastened to one another in an airtight manner.

6. The device according to claim 1,
characterised in that,
the ventilating hose (5) is enclosed by the exhalation hose (6) and the latter, by means of a valve, passes the air flow (12) pressed out by the ventilating device (2) into the ventilating hose (6) and then into the respiratory tract therapy device (31).

7. The device according to claim 6,
characterised in that,
the hose (33) of the respiratory tract therapy device (31) is closed by the exhalation pressure of the ventilating device (2) in the manner of a valve, and in that the used breathing air (13) is conducted laterally beside this hose (33) in the direction of the ventilating device (2).

8. The device according to claim 1,
characterised in that,
a bypass line (18), into which a valve (19) is inserted, is provided parallel to the respiratory tract therapy device (31), and in that the bypass line (18) is opened or closed by the valve (19) and the respiratory tract therapy device (31) is connected to or separated from the ventilating device (2).

9. The device according to claim 8,
characterised in that,
the valve (19) forms a type of branch for the oxygen flow from the ventilating device (22) to the living being (21), and in that the valve (19) can be adjusted manually from the outside.
